(19) United States
(12) Patent Application Publication
(10) Pub. No.: US 2025/0255786 A1
(43) Pub. Date: Aug. 14, 2025

(54) COMPOSITION AND METHOD FOR A ROOT CANAL FILLING

(71) Applicant: Rutgers, The State University of New Jersey, New Brunswick, NJ (US)

(72) Inventor: Mohammad Ali Saghiri, New Brunswick, NJ (US)

(21) Appl. No.: 19/077,605

(22) Filed: Mar. 12, 2025

Related U.S. Application Data
(63) Continuation-in-part of application No. PCT/US2023/032861, filed on Sep. 15, 2023.
(60) Provisional application No. 63/407,018, filed on Sep. 15, 2022.

(51) Int. Cl. A61K 6/893; A61K 6/17; A61K 6/831

(52) U.S. Cl. CPC A61K 6/893; A61K 6/17; A61K 6/831

(57) ABSTRACT

A composition is provided. The composition comprises a polyol part. The polyol part comprises a catalyst, a defoamer, and modified nanoparticles. The catalyst comprises at least one of a base catalyst, a metal catalyst, a quaternary ammonium catalyst, a phosphorus-based catalyst, or any combination thereof. The composition is an elastomeric polyurethane sealer.

### CROSS REFERENCE TO RELATED APPLICATION

This patent application is a Continuation-in-part Application relating to and claiming the benefit of commonly-owned, co-pending PCT International Application No. PCT/US2023/032861, filed Sep. 15, 2023, which claims priority to and the benefit of U.S. Provisional Patent Application No. 63/407,018, filed Sep. 15, 2022, the contents of each of the foregoing are herein incorporated by reference in their entirety.

### FIELD

Some embodiments of the present disclosure relate to compositions and methods for use in endodontic treatment and, more particularly, compositions for root canal filling and methods for use of same.

### BACKGROUND

Endodontic treatment or root canal therapy (RCT) is a commonly performed tooth treatment in the field of dentistry. Conventionally, RCT is directed towards the treatment of a tooth inner space, which contains a soft tissue known as “dental pulp tissue”, and then filling the inner space with biocompatible materials. After the removal of dental pulp from, and after the cleaning, shaping and/or irrigation of, the tooth root canal space, the cleaned, disinfected, and shaped root canal space is dried out and then filled by biocompatible materials.

### BRIEF SUMMARY

Some embodiments relate to a composition. In some embodiments, the composition comprises a polyol part. In some embodiments, the polyol part comprises a catalyst. In some embodiments, the catalyst comprises at least one of a base catalyst, a metal catalyst, a quaternary ammonium catalyst, a phosphorus-based catalyst, or any combination thereof. In some embodiments, the polyol part comprises a defoamer. In some embodiments, the polyol part comprises modified nanoparticles. In some embodiments, the composition is an elastomeric polyurethane sealer.

Some embodiments relate to a composition. In some embodiments, the composition comprises a polyol part. In some embodiments, the polyol part comprises a catalyst. In some embodiments, the catalyst comprises at least one of a base catalyst, a metal catalyst, a quaternary ammonium catalyst, a phosphorus-based catalyst, or any combination thereof. In some embodiments, the polyol part comprises a defoamer. In some embodiments, the polyol part comprises modified nanoparticles. In some embodiments, the composition comprises a diisocyanate part. In some embodiments, the diisocyanate part and the polyol part are present at a 1:1 molar ratio. In some embodiments, the composition is an elastomeric polyurethane sealer.

### BRIEF DESCRIPTION OF THE DRAWINGS

FIG. 1A is a schematic views of root canal spaces in a tooth, according to embodiments of the present disclosure.

FIG. 1B is a top cross-sectional views of root canals which illustrate complex and irregular tooth root canal patterns, according to embodiments of the present disclosure.

FIG. 2 is a schematic view of filled/obturated tooth root canal spaces, according to embodiments of the present disclosure.

FIG. 3A is a perspective view of a prototype of an EPS sealer, according to embodiments of the present disclosure.

FIG. 3B is a perspective view of a pre-prototype of EPS, according to embodiments of the present disclosure.

FIG. 4 is a box plot graph of the means and standard deviations of surface porosity of various sealers, according to embodiments of the present disclosure.

FIG. 5A is a box plot graph of the means and standard deviations of cell viability in MTS for various sealers at 24 hours, according to embodiments of the present disclosure.

FIG. 5B is a box plot graph of the means and standard deviations of cell viability in MTS for various sealers at 48 hours, according to embodiments of the present disclosure.

FIG. 5C is a box plot graph of the means and standard deviations of cell viability in MTS for various sealers at 72 hours, according to embodiments of the present disclosure.

FIG. 5D is a box plot graph of the means and standard deviations of cell viability in MTS for various sealers at 96 hours, according to embodiments of the present disclosure.

FIG. 6 is a box plot graph of the means and standard deviations of CNV surface area (μm²) of various sealers, according to embodiments of the present disclosure.

FIG. 7 is a box plot graph of the means and standard deviations of penetration depth (μm) for various sealers, according to embodiments of the present disclosure.

FIG. 8A is an SEM micrograph showing the surface porosity of AH Plus, according to embodiments of the present disclosure.

FIG. 8B is an SEM micrograph showing the surface porosity of Sure Seal, according to embodiments of the present disclosure.

FIG. 8C is an SEM micrograph showing the surface porosity of PES, according to embodiments of the present disclosure.

FIG. 8D is an SEM micrograph showing the surface porosity of EPS, according to embodiments of the present disclosure.

FIG. 9A is a box plot graph of means and standard deviations of flow diameters of various sealers, according to embodiments of the present disclosure.

FIG. 9B is a box plot graph of means and standard deviations of setting times of various sealers, according to embodiments of the present disclosure.

FIG. 9C is a box plot graph of means and standard deviations of radiopacity of various sealers, according to embodiments of the present disclosure.

FIG. 9D is a box plot graph of means and standard deviations of flow surface area of various sealers, according to embodiments of the present disclosure.

FIG. 10 is a graph depicting in-vitro weight loss of various sealers during a test period of 16 weeks using an accelerated aging model, according to embodiments of the present disclosure.

FIG. 11 is a schematic view of a multi-barrel syringe containing embodiments of a first fluid and a second fluid contained in respective barrels, according to embodiments of the present disclosure.

FIG. 12 is a flowchart for a process according to an embodiment, according to embodiments of the present disclosure.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

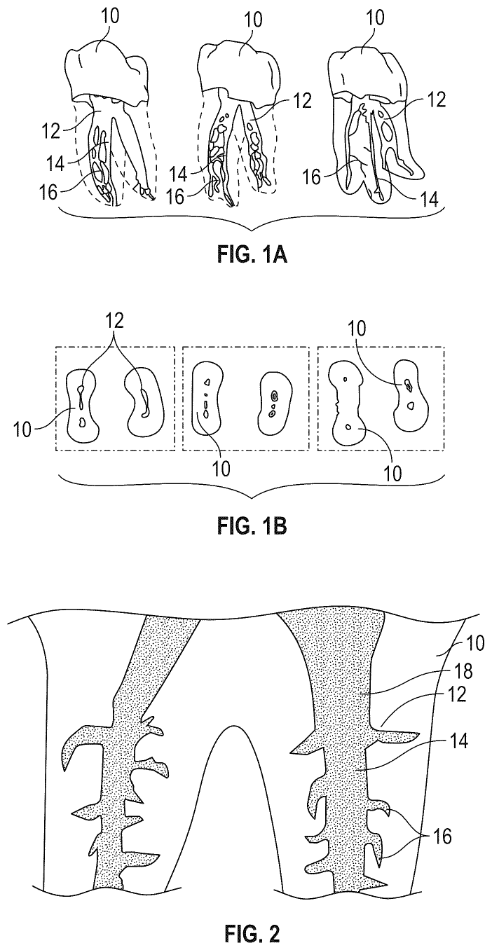

FIGS. 1A and 1B show exemplary views of a tooth 10 having complex and irregular patterns of tooth root canal spaces 12 according to an embodiment. Tooth root canal spaces 12 may have very complex structures and irregular patterns, as they can include main canals 14 and accessory canals 16. Thus, cleaning, disinfecting and filling such irregular patterns may pose difficulties.

In an embodiment, a filling material 18 comprises at least one expandable material. The at least one expandable material of the filling material 18 may include at least one of: a swellable material, a foamable material, the like, or any combination thereof. In some embodiments, the expandable material of the filling material 18 may be a crosslinkable material that expands upon crosslinking. In some embodiments, the crosslinking may be performed in situ. In an embodiment, the filling material 18 is configured to expand so as to fill most, substantially all of, or all of the main canals 14 and accessory canals 16 of tooth root canal spaces 12. In an embodiment, the filling material 18 comprises at least one biocompatible material. In an embodiment, the at least one at least one biocompatible material is an expandable material.

FIG. 2 shows a schematic view of an embodiment of a filled or obturated root canal space 12 showing main and accessory canals 14, 16 being filled with the filling material 18.

In some embodiments the expandable material of the filling material 18 expands using isocyanate chemistry (i.e., the chemistry of a material that includes at least one isocyanate group). For instance, in one embodiment, the isocyanate chemistry may be utilized to induce cross-linking of the expandable material of the filling material 18. In one embodiment, the isocyanate chemistry may be utilized to induce foaming of the expandable material of the filling material 18.

Materials including isocyanate groups can become unstable when exposed to various conditions including, but not limited to: the presence of, water, the presence of moisture, the presence of other compounds, and the like. Therefore, various conditions can lead to the decomposition of the isocyanate groups, thereby cross-linking one or more polymers to which the isocyanate groups are attached and releasing carbon dioxide (CO₂) gas.

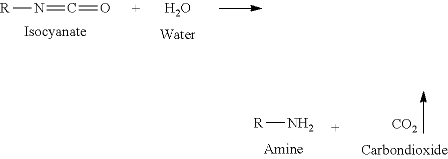

The CO₂ gas that is released form pores in some embodiments. In some embodiments, the production or releasing of the CO₂ gas is controlled, thereby controlling the formations of the pores in the biocompatible cross-linked polymers. By controlling the production or releasing of the CO₂ gas, the three-dimensional structure and thereby its physical properties (such as for example, compression and/or expansion properties) are also controlled to a specific and desired amount. Further, the controlling of the CO₂ gas that are exposed to nearby tissue can beneficially minimize the pH change effect in the tissue.

For example, physiologically normal intracellular pH is most commonly between 7.0 and 7.4, though there is variability between tissues (e.g., mammalian skeletal muscle tends to have a pH of 6.8-7.1). Generally, dental infections have some infected tissue which has acidic pH. For example, the pH of pus from a periapical abscess of infected tissue can have a range between 6.0 and 7.3. Therefore, a biocompatible material that does not alter the surrounding tissue pH can be beneficial and advantageous. The embodiments of the biocompatible polymers disclosed herein can be configured to (e.g., controlled) to release low amount of CO₂ (e.g., less than 7% of weight). Accordingly, the embodiments of the biocompatible polymers and methods disclosed herein have substantial benefits and advantages over conventional materials and methods.

A non-limiting exemplary mechanism by which a material containing an isocyanate

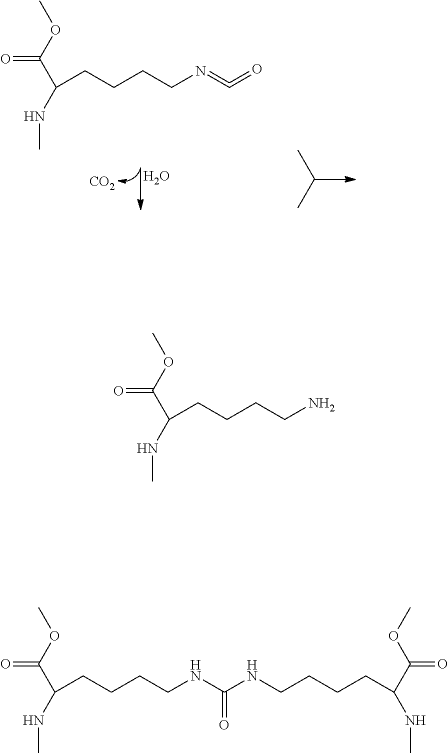

group is crosslinked is shown below. The nonlimiting exemplary mechanism below may be referred to as the “lysine model” of isocyanate crosslinking.

In an embodiment, the filling material 18 includes multiple expandable materials. In some embodiments, the multiple expandable materials are configured to expand upon cross-linking.

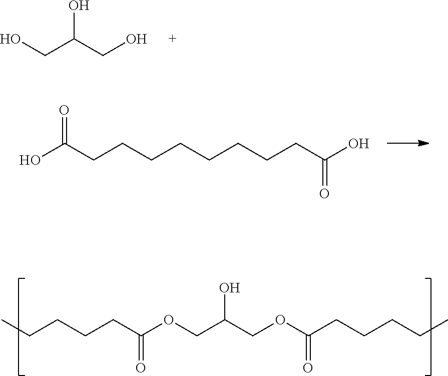

In an embodiment, the use of multiple expandable materials ensures that the filling material 18 is stable in water and saline.

In an embodiment, an expandable material of the filling material 18 comprises at least one condensation polymer. In an embodiment, the at least one condensation polymer comprises poly glycerol-sebacate (“PGS”). In an embodiment, the at least one condensation polymer is formed by the condensation polymerization of glycerol and sebacic acid. A non-limiting exemplary synthesis pathway by which glycerol and sebacic acid are reacted by condensation polymerization to form PGS is shown below:

In an embodiment, a polymer of the expandable material of the filling material 18 comprises at least one of: poly (lactic acid) (PLA), poly (glycolic acid) (PGA), at least one polymer from the polycaprolactone (PCL) class of polymers and their copolymers (e.g., poly (lactate-co-caprolactone) or poly (glycolate-caprolactone)), or any combination thereof. In an embodiment, copolymerization of at least one lactide, glycolide, or caprolactone monomer present on at least one polymer of the expandable material of the filling material 18 described herein, in various ratios, can yield materials with a wide range of mechanical properties, thermal characteristics and degradation times. In an embodiment, a structure of an exemplary PLA/PGA/PCL copolymer (and associated properties such as molecular weight) can be tailored by adjusting, for example, a type of initiator used, a molar ratio of the initiator to the at least one monomer unit, or any combination thereof.

A non-limiting synthesis pathway for poly(glycolide-co-caprolactone) (PGCL) according to one exemplary embodiment is shown below. In the non-limiting pathway below, pentaerythritol is used as an initiator to form 4-armed, branched structures.

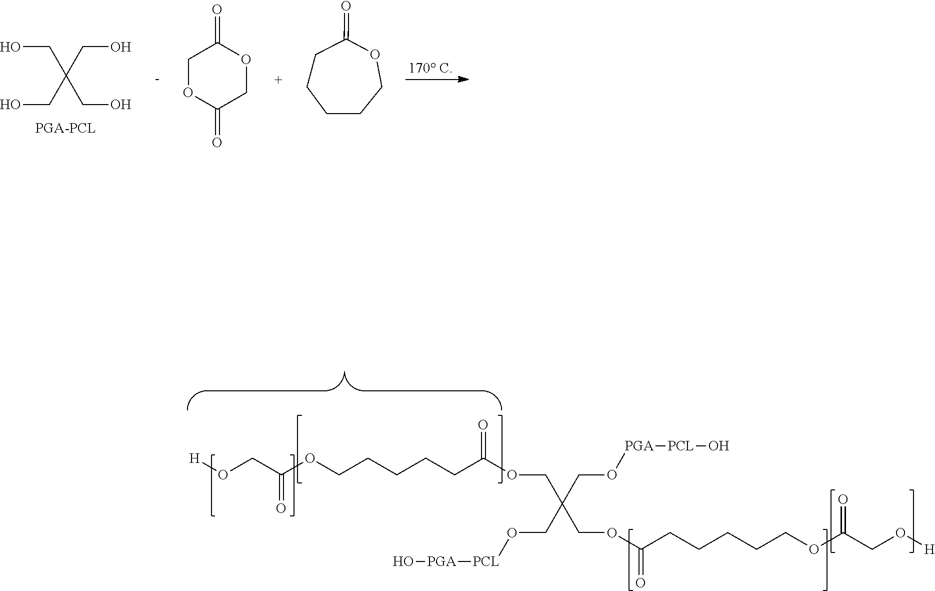

In an embodiment, the at least one polymer of the expandable material of the filling material 18 (or the compounds used to make the polymer of the expandable material of the filling material 18) may comprise one or more pendant hydroxyl groups. In an embodiment, the hydroxyl groups may serve, for example, as sites at which pendant groups are attached to the at least one polymer. In an embodiment, glycerol and sebacic acid both contain pendant hydroxyl groups that may be used to impart a desired functionality to PGS. In an embodiment, the filling material 18 may include at least one radiopaque material. In an embodiment, the at least one radiopaque material may include at least one of the following: gold, platinum, tungsten, platinum-tungsten, palladium, iridium, platinum-iridium, rhodium, tantalum, barium sulfate, bismuth subcarbonate, bismuth oxychloride, bismuth trioxide, the like (e.g., a radiopaque metal, a radiopaque alloy, or a radiopaque ceramic), or any combination thereof.

In an embodiment, the filling material 18 may include at least one biostable material. In an embodiment, the at least one biostable material prevents the degradation of the filling material 18 by one or more endogenous enzymes. In a non-limiting exemplary embodiment, the biostable material includes at least one biostable metal oxide, such as for example but not limited to one or more of titanium oxide, ruthenium oxide, and iridium oxide.

In an embodiment, porous polyurethane scaffolds are synthesized when the (PCLG) triol and isocyanate react, with CO₂ acting as a blowing agent to create pores.

In an embodiment, at least one expandable material of the filling material 18 comprises at least one polymer foam. In some embodiments, the polymer foam incudes a polymer material including a compound of Formula [A]:

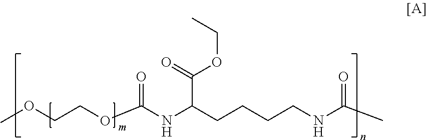

According to an embodiment, a compound of Formula [B] can be prepared and be mixed with a compound of Formula [C]:

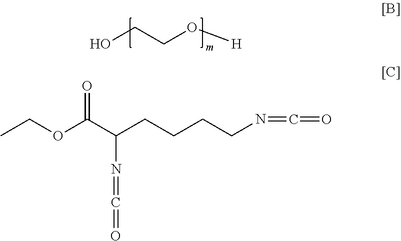

The mixing of the compound [B] and compound [C] leads to polymerization to form the compound of Formula [A]. In an embodiment, the material that includes the compound of Formula [A] has a density of 0.10-0.40 g/cm³.

In some embodiments, a number of units “m” in the polymer comprising the Formula [A] is an integer in a range of 1 to 100 million.

In some embodiments, a number of monomer units “n” in the polymer comprising the Formula [A] is an integer in a range of 2 to 100 million. In some embodiments, the number of monomer units “n” in the polymer comprising the Formula [A] is an integer in a range of 2 to 50 million. In some embodiments, the number of monomer units “n” in the polymer comprising the Formula [A] is an integer in a range of 2 to 10 million. In some embodiments, the number of monomer units “n” in the polymer comprising the Formula [A] is an integer in a range of 2 to 5 million. In some embodiments, the number of monomer units “n” in the polymer comprising the Formula [A] is an integer in a range of 2 to 1 million. In some embodiments, the number of monomer units “n” in the polymer comprising the Formula [A] is an integer in a range of 2 to 100,000. In some embodiments, the number of monomer units “n” in the polymer comprising the Formula [A] is an integer in a range of 2 to 10,000. In some embodiments, the number of monomer units “n” in the polymer comprising the Formula [A] is an integer in a range of 2 to 1000. In some embodiments, the number of monomer units “n” in the polymer comprising the Formula [A] is an integer in a range of 2 to 500. In some embodiments, the number of monomer units “n” in the polymer comprising the Formula [A] is an integer in a range of 2 to 100.

In some embodiments, the number of monomer units “n” in the polymer comprising the Formula [A] is an integer in a range of 100 to 100 million. In some embodiments, the number of monomer units “n” in the polymer comprising the Formula [A] is an integer in a range of 1000 to 100 million. In some embodiments, the number of monomer units “n” in the polymer comprising the Formula [A] is an integer in a range of 10,000 to 100 million. In some embodiments, the number of monomer units “n” in the polymer comprising the Formula [A] is an integer in a range of 10,000 to 100 million. In some embodiments, the number of monomer units “n” in the polymer comprising the Formula [A] is an integer in a range of 1 million to 100 million. In some embodiments, the number of monomer units “n” in the polymer comprising the Formula [A] is an integer in a range of 10 million to 100 million. In some embodiments, the number of monomer units “n” in the polymer comprising the Formula [A] is an integer in a range of 50 million to 100 million.

In some embodiments, the composition comprising Formula [A] is a maxillofacial filler.

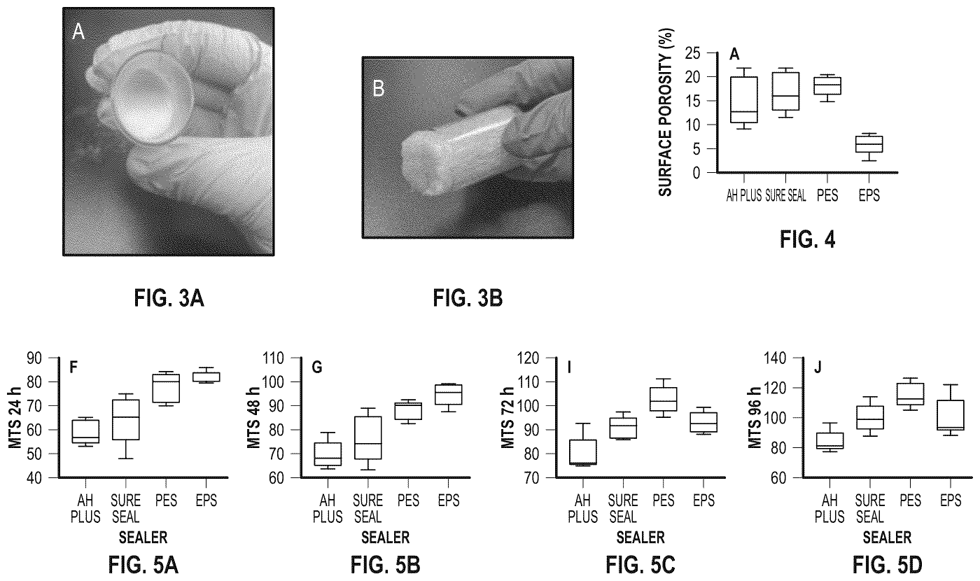

Turning to FIGS. 3A and 3B, a sealer is disclosed according to embodiments of the present disclosure. In some embodiments, the sealer is a polymeric sealer. In some embodiments, the sealer is an elastomeric polyurethane sealer (EPS). FIG. 3A depicts a prototype of an EPS sealer according to embodiments of the present disclosure. FIG. 3B depicts a pre-prototype of EPS according to embodiments of the present disclosure. In some embodiments, the EPS formulation includes a polyol part including a catalyst, a defoamer, modified nanoparticles. In some embodiments, the EPS formulation includes a diisocyante part.

In some embodiments, the polyol part of the elastomeric polyurethane sealer composition can influence the mechanical properties and biocompatibility of the composition. In some embodiments, the polyol part can be selected to meet specific clinical requirements, thereby enhancing the performance in endodontic treatments. In some embodiments, the polyol part comprises a hydroxyl terminated polyol. In some embodiments, the hydroxyl terminated polyol can comprise at least one of a polyether polyol, a polycaprolactone polyol (PCL), a polytetrahydrofuran (PTHF), a polyester polyol, a polycarbonate polyol, or any combination thereof.

In some embodiments, the hydroxyl terminated polyol includes hydroxyl terminated polybutadiene (HTPB). In some embodiments, the HTPB can enhance the flexibility and hydrophobicity of the sealer. In some embodiments, the hydrophobic nature of HTPB can reduce moisture diffusion into the dentinal tubules, thereby controlling the expansion of the sealer and minimizing the release of carbon dioxide during the curing process. In some embodiments, the HTPB comprises a HTPB with different molecular weights or mircrostructures, such as varying cis/trans ratios to modify flexibility and reactivity.

In some the polyether polyols can enhance compatibility and mechanical properties of the composition. In some embodiments, the polyether polyols comprises at least one of polyethylene glycol (PEG), polypropylene glycol (PPG), or any combination thereof.

In some embodiments, the PCL can enhance the mechanical properties, flexibility, and abrasion resistance of the composition.

In some embodiments, the PTHF can enhance elasticity and provide improved low-temperature performance of the composition.

In some embodiments, the polyester polyol can provide enhance mechanical properties and lower hydrolytic stability of the composition.

In some embodiments, the polycarbonate poly can provide resistance to hydrolysis, UV light, and heat for the composition.

In some embodiments, the choice of catalyst can optimize the performance of the composition, in terms of the expansion characteristics and compatibility with dental tissues. In some embodiments, the catalyst comprises at least one of a base catalyst, a metal catalyst, a quaternary ammonium catalyst, a phosphorus-based catalyst, or any combination thereof.

In some embodiments, the composition utilizes a base catalyst to facilitate the polymerization process. In some embodiments, the base catalyst can provide rapid and controlled curing process to achieve the mechanical properties and biocompatibility of the sealer. In some embodiments, the base catalyst comprises at least one of a guanidine-based catalyst, an amidine-based catalyst, or any combination thereof.

In some embodiments, the guanidine-based catalyst can maintain the basicity and catalytic profile of the composition. In some embodiments, the guanidine-based catalyst at least one of tetramethylguanidine or 1,1,3,3-tetramethylguanidine, pentamethylguanidine, or any combination thereof.

In some embodiments, the composition comprises 0.3% to 0.7% by weight of the catalyst based on a total composition, or any range or subrange between 0.3% and 0.7%. For example, in some embodiments, the composition comprises 0.4% to 0.6%, 0.3% to 0.6%, 0.3% to 0.5%, 0.3% to 0.4%. 0.4% to 0.7%, 0.5% to 0.7%, or 0.6% to 0.7% by weight of the catalyst based on the total composition.

In some embodiments, the catalyst is pentamethylguanidine at 0.5 wt. %. In some embodiments, the catalyst is pentamethylguanidine at 0.3 wt. % to 0.7 wt. %. In some embodiments, the catalyst is pentamethylguanidine at 0.35 wt. % to 0.7 wt. %. In some embodiments, the catalyst is pentamethylguanidine at 0.4 wt. % to 0.7 wt. %. In some embodiments, the catalyst is pentamethylguanidine at 0.45 wt. % to 0.7 wt. %. In some embodiments, the catalyst is pentamethylguanidine at 0.5 wt. % to 0.7 wt. %. In some embodiments, the catalyst is pentamethylguanidine at 0.55 wt. % to 0.7 wt. %. In some embodiments, the catalyst is pentamethylguanidine at 0.6 wt. % to 0.7 wt. %. In some embodiments, the catalyst is pentamethylguanidine at 0.65 wt. % to 0.7 wt. %.

In some embodiments, the catalyst is pentamethylguanidine at 0.3 wt. % to 0.65 wt. %. In some embodiments, the catalyst is pentamethylguanidine at 0.3 wt. % to 0.6 wt. %. In some embodiments, the catalyst is pentamethylguanidine at 0.3 wt. % to 0.55 wt. %. In some embodiments, the catalyst is pentamethylguanidine at 0.3 wt. % to 0.5 wt. %. In some embodiments, the catalyst is pentamethylguanidine at 0.3 wt. % to 0.45 wt. %. In some embodiments, the catalyst is pentamethylguanidine at 0.3 wt. % to 0.4 wt. %. In some embodiments, the catalyst is pentamethylguanidine at 0.3 wt. % to 0.35 wt. %.

In some embodiments, the catalyst is pentamethylguanidine at 0.4 wt. % to 0.6 wt. %. In some embodiments, the catalyst is pentamethylguanidine at 0.45 wt. % to 0.55 wt. %. In some embodiments, the catalyst is pentamethylguanidine at 0.5 wt. % to 0.65 wt. %. In some embodiments, the catalyst is pentamethylguanidine at 0.35 wt. % to 0.45 wt. %. In some embodiments, the catalyst is pentamethylguanidine at 0.4 wt. % to 0.5 wt. %. In some embodiments, the catalyst is pentamethylguanidine at 0.4 wt. % to 0.65 wt. %.

In some embodiments, the amidine-based catalyst can be used in the composition if the amidine-based catalyst can provide comparable reactivity without adverse side effects.

In some embodiments, the metal-based catalyst can be used in the composition if optimized to balance catalytic efficiency with safety and regulatory compliance. In some embodiments, the metal-bast catalyst comprises at least one of dibutyltin dilaurate, bismuth carboxylate, or any combination thereof.

In some embodiments, the quaternary ammonium catalyst comprises tetraethylammonium bromide. In some embodiments, the quaternary ammonium catalyst is not biocompatible.

In some embodiments, the phosphorus-based catalyst comprises tris(dimethylaminomethyl)phenol.

In some embodiments, the composition includes a defoamer to control the expansion properties of the elastomeric polyurethane sealer. In some embodiments, the defoamer is configured to reduce the formation of foam during the polymerization process. In some embodiments, the reduction in foam formation is important for ensuring the uniformity and consistency of the sealer, thereby enhancing the mechanical properties and biocompatibility of the sealer. In some embodiments, the defoamer comprises at least a silicone-based defoamer, a non-silicone based defoamer, or any combination thereof.

In some embodiments, the composition comprises 0.1% to 0.3% by weight of the defoamer based on a total composition, or any range or subrange between 0.1% and 0.3%. For example, in some embodiments, the composition comprises 0.2% to 0.3% or 0.1% to 0.2% by weight of the defoamer based on a total composition.

In some embodiments, the defoamer can be a silicone-based defoamer. In some embodiments, the silicone-based defoamer can disrupt the surface tension of bubbles, allowing the bubbles to collapse and preventing excessive expansion of the composition. In some embodiments, the silicone-based defoamer comprises Antifoam A concentrate. In some embodiments, the silicone-based defoamer comprises variants of a silicone-based defoamer with different alkyl chain modification to adjust defoaming properties.

In some embodiments, the silicone-based defoamer is Antifoam A concentrate. In some embodiments, the defoamer is 100% active silicone polymer. In some embodiments, the defoamer is present at 0.2 wt. %. In some embodiments, the defoamer is Antifoam A concentrate at 0.1 wt. % to 0.3 wt. %. In some embodiments, the defoamer is Antifoam A concentrate at 0.15 wt. % to 0.3 wt. %. In some embodiments, the defoamer is Antifoam A concentrate at 0.2 wt. % to 0.3 wt. %. In some embodiments, the defoamer is Antifoam A concentrate at 0.25 wt. % to 0.3 wt. %.

In some embodiments, the defoamer is Antifoam A concentrate at 0.1 wt. % to 0.25 wt. %. In some embodiments, the defoamer is Antifoam A concentrate at 0.1 wt. % to 0.2 wt. %. In some embodiments, the defoamer is Antifoam A concentrate at 0.1 wt. % to 0.15 wt. %.

In some embodiments, the non-silicone based defoamer comprises at least one of a polyether-based defoamer, a fluorosilicone-based defoamer, a mineral oil based defoamer, a fatty acid-based defoamer, an alcohol-based defoamer, an ester-based defoamer, or any combination thereof. In some embodiments, the polyether-based defoamer and fluorosilicone-based defoamer can provide distince expansion control properties to the composition. In some embodiments, the mineral oil based defoamer and the fatty acid-based defoamer can be used in combination to enhance performance of the composition.

In some embodiments, the modified nanoparticles comprises fatty-acid functionalized particles. In some embodiments, the fatty-acid functionalized particles comprises 5 wt % stearic acid, palmitic acid, and oleic acid. In some embodiments, the palmitic acid and the oleic acid can adjust interfacial properties and polymer compatibility in the composition.

In some embodiments, the modified nanoparticles include 5 wt. % stearic acid to improve their dispersion and the adhesion between the filler and polymer matrix.

In some embodiments, the modified nanoparticles comprises at least one of a carboxyl modified nanoparticles, an amine modified nanoparticles, a thiol-functionalized nanoparticles, or any combination thereof for enhanced reactivity or mechanical performance.

In some embodiments, the modified nanoparticles comprise surface hydroxyl groups configured to react with diisocynates.

In some embodiments, the composition comprises a diisocyanate part. In some embodiments, the diisocyanate part comprises at least a bio-based diisocyanate, a non-aromatic diisocyanate, an aromatic diisocyanate, or any combination thereof.

In some embodiments, the bio-based diisocyanate comprises L-lysine diisocyanate (LDI). In some embodiments, the bio-based diisocyanate comprises diisocyanates derived from amino acids or renewable sources. For example, in some embodiments, the bio-based diisocyanate comprises amino acid-based isocyanates, lignin-based isocyanates, and vegetable oil-based isocyanates.

In some embodiments, the non-aromatic diisocyanate comprises aliphatic diisocyanates and cycloaliphatic diisocyanates. In some embodiments, the cycloaliphatic diisocyantes comprises hexamethylene diisocyanate (HDI) and isophorone diisocyanate (IPDI).

In some embodiments, the aromatic diisocyanates comprises methylene diphenyl diisocyanate (MDI) and toluene diisocyanate (TDI).

In some embodiments, the diisocyanate part comprises a moisture scavenger.

n some embodiments, the polymeric sealer prepared by mixing the polyol and diisocyanate parts based on a 1:1 molar ratio of diisocyanate and HTPB.

In some embodiments, the EPS formulation is capable of controlling the amount of foaming by improving the hydrophobicity of the material through HTPB, thereby reducing the diffusion of moisture into the dentinal tubules and the subsequent release of CO₂. In addition to controlling the amount of expansion, HTPB has a structure compatible with gutta-percha, preventing any phase separation in the structure of these two compounds and indicating the likelihood of using the EPS formulation in combination with other filling materials. In some embodiments, surface modification of essential fillers by the EPS formulation will ensure proper distribution of the fillers to reduce water absorption and prevent their agglomeration. Further, unmodified nanoparticles contain surface hydroxyl groups that may react with diisocyanates in the polymerization reaction as evidenced in the first formulation below.

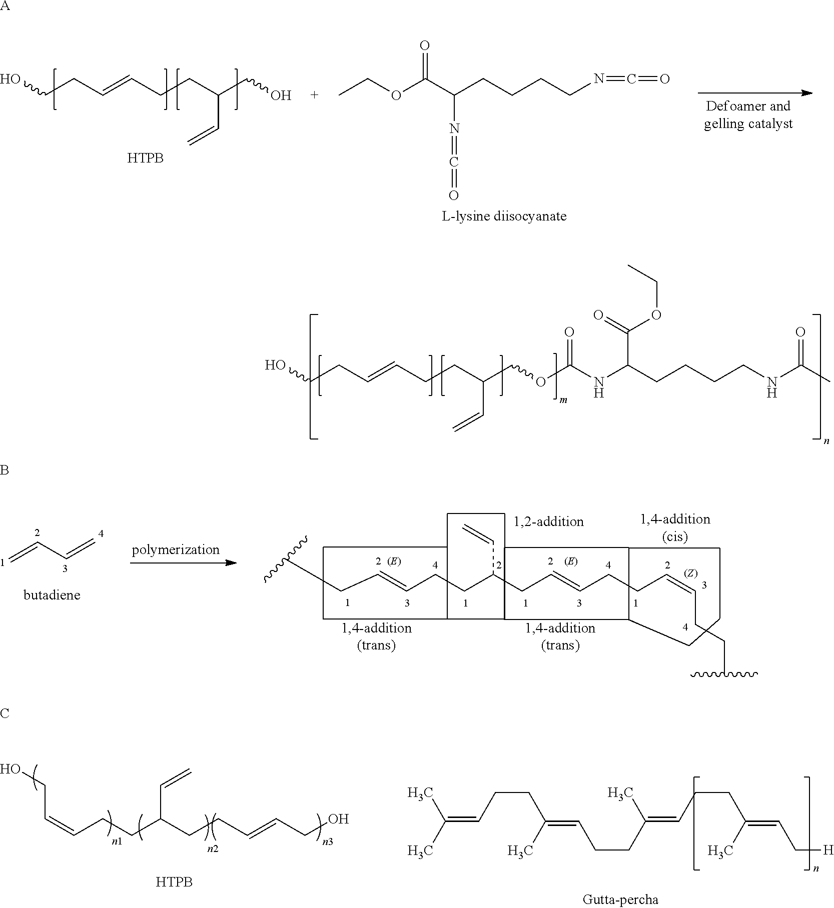

### Example 1

Testing was performed to evaluate the biocompatibility, porosity, and penetration ability of our newly developed expandable sealer. Testing of the sealers AH Plus, Sure Seal, polyurethane expandable sealer (PES), and EPS in terms of their cytotoxicity was performed according to ISO 10993-5. MTT assays showed that both formulations of EPS were significantly less cytotoxic in all time intervals, possibly because of its less release of toxic by-products in comparison with the other tested sealers.

The second model utilized in this study was the laser-induced mouse CNV model. ImageJ software was used to measure the total area (in μm2) of CNV associated with each spot hit by the laser in the mice. Results of the CNV assay showed that both formulations of EPS were more proangiogenic than other tested sealers, which may favor apical tissue regeneration.

Furthermore, results showed that the penetration depth of EPS was significantly higher than AH Plus and Sure Seal. The deeper penetration may not only be due to lower film thickness, viscosity, and surface tension of EPS but also due to the superior expandability of EPS caused by the production of CO2 that pushes the sealer into the dentinal tubules. SEM micrographs showed that the surface porosity was significantly higher in AH Plus, Sure Seal, and PES compared to EPS.

In the next set of testing, evaluations of the sealers AH Plus, EndoSequence BC, as well as PES and EPS in terms of flow, radiopacity, and setting time were performed according to ISO 6876:2012. Results showed that the highest surface area and diameter of flow were detected in EPS, allowing the filling material to penetrate the dentinal tubules and irregularities of the root canal space significantly more than other tested sealers. The shorter setting time of EPS might be explained by the rapid reaction of these sealers, which was more accelerated for the newly developed formula. It can be hypothesized that by increasing the expansion rate, the setting time is reduced. Moreover, the reduced radiopacity of PES compared to AH Plus might be explained by differences in the ratio of radiopaque agents and/or the nano form of zirconium oxide used in the composition of PES.

Cytotoxicity testing was also performed according to ISO 10993-5. Results showed that the EPS formulation was more proangiogenic than other tested sealers, which may favor apical tissue regeneration.

Testing was also performed for the improved polymer radiopacity and catalyst ratios in the filler ingredients of the EPS formulation (including nano zinc oxide, zirconium oxide nanoparticles, and nano calcium tungstate). Results showed that the EPS formulation had a higher radiopacity compared to EndoSequence BC and PES.

FIG. 4 depicts box plots of the means and standard deviations of surface porosity for the various sealers.

FIGS. 5A-5D depict box plots of the means and standard deviations of cell viability in MTS for various sealers are shown at: 24 hours (FIG. 5A), 48 hours (FIG. 5B), 72 hours (FIG. 5C) and 96 hours (FIG. 5D).

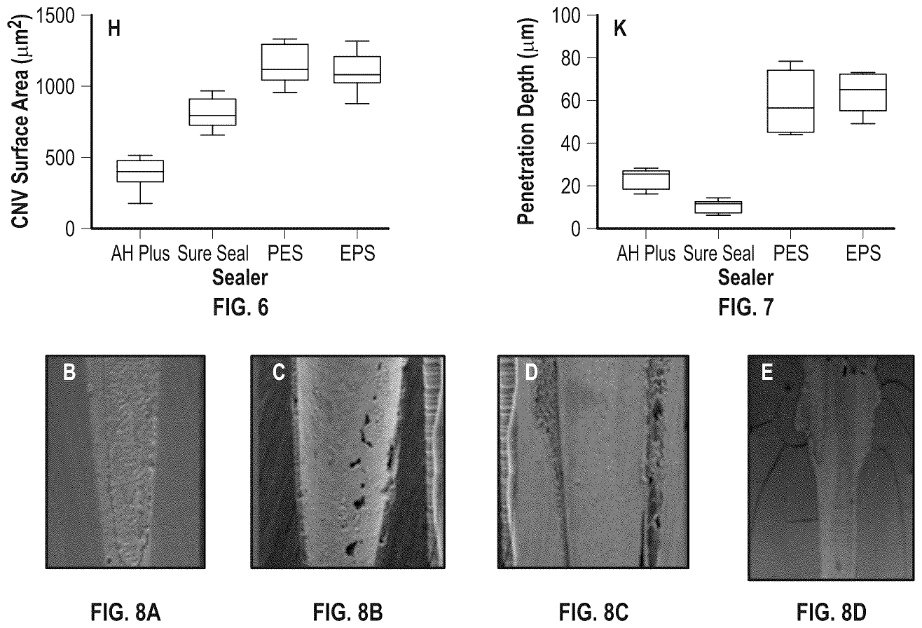

FIG. 6 depicts box plots of the means and standard deviations of CNV surface area (μm²) of various sealers.

FIG. 7 depicts box plots of the means and standard deviations of penetration depth (μm) for various sealers.

FIGS. 8A-8D depict SEM micrographs showing surface porosity at the gutta-percha and root canal dentin interface. FIG. 8A depicts the porosity visible in AH Plus. FIG. 8B depicts gaps visible for Sure Seal due to shrinkage. FIG. 8C depicts that porosity is visible for PES due to polyethylene glycol. FIG. 8D depicts that no porosity is visible for EPS due to HTPB.

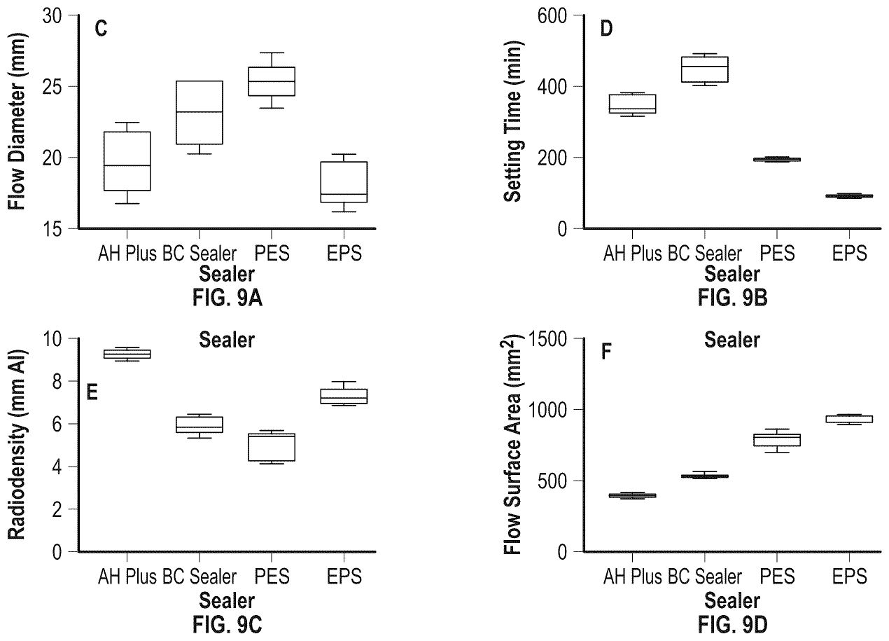

FIGS. 9A-9D depict box plots of means and standard deviations of experimental sealer groups. Specifically, FIG. 9A depicts flow diameters (mm) of various sealers; FIG. 9B depicts setting time (min) of various sealers; FIG. 9C depicts radiopacity (mm Al) of various sealers; and FIG. 9D depicts flow surface area (mm²) of various sealers.

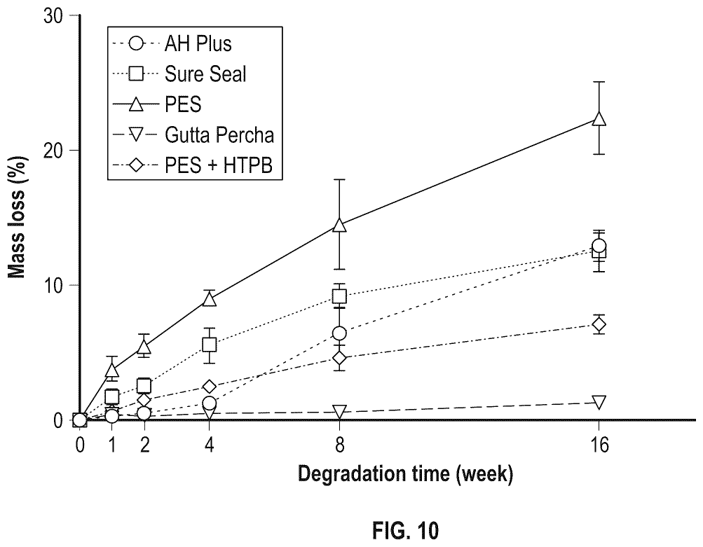

FIG. 10 depicts in-vitro weight loss of various sealers during a test period of 16 weeks using an accelerated aging model. An EPS formulation, according to embodiments of the present disclosure, was found to be significantly more stable than all other sealers. Gutta-percha was used as a control.

Example Method of Use 1: In an exemplary non-limiting embodiment, the filling material 18 can be prepared in a dental operating room setting by the following steps:
1. Adding a hardener to isocyanate in a sterile canister.
2. Inserting the canister into a fixture loading tray.
3. Lowering an impeller into the canister at a pre-set mixing height.
4. Mixing the isocyanate and hardener mixture in the canister for about 40 seconds at about 11,000 RPM.
5. Withdrawing the shaft from the canister.
6. Removing the canister from loading tray.
7. Injecting polyurethane into the mixed isocyanate and hardener mixture.

Example Method of Use 2: In another exemplary non-limiting embodiment, the filling material 18 can be prepared in a dental operating room setting by the following steps:
1. Adding a hardener to isocyanate in a sterile canister.
2. Inserting the canister into a fixture loading tray.
3. Lowering an impeller into the canister at a pre-set mixing height.
4. Mixing the isocyanate and hardener mixture in the canister for 30-50 seconds at 10,000 to 12,000 RPM. For example, mixing the isocyanate and hardener mixture in the canister for 40 seconds at 11,000 RPM.
5. Withdrawing the shaft from the canister.
6. Removing the canister from loading tray.
7. Injecting biopolymer into the isocyanate and hardener mixture.

In an embodiment, the resulting mixture results in middle or low level porous and biocompatible biopolymers that can be use as the root filing material 18.

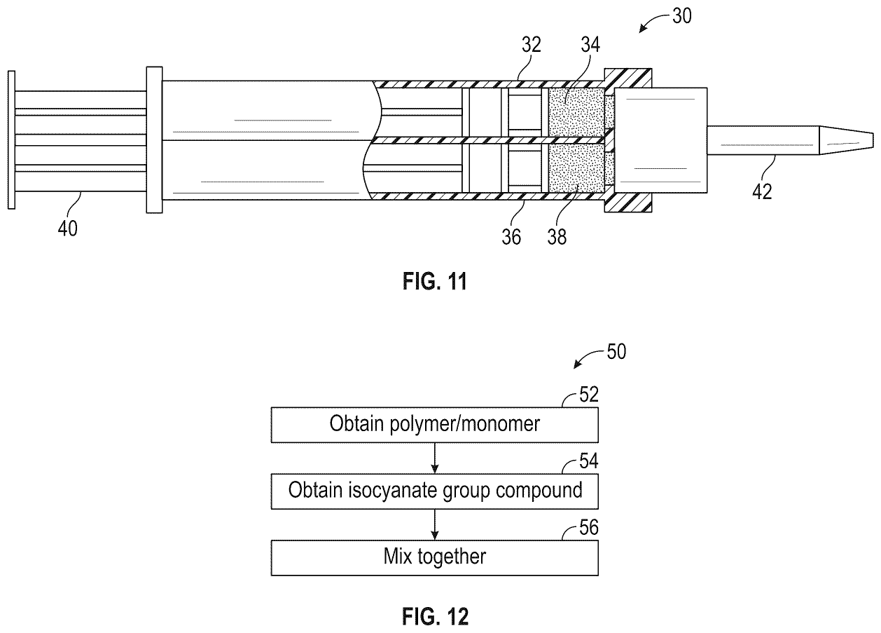

FIG. 11 shows a nonlimiting exemplary embodiment of a multi-barrel syringe 30 (e.g., a double barrel syringe) comprising a first barrel chamber 32 containing a first fluid 34, and a second barrel chamber 36 containing a second fluid 38.

In an embodiment, the first fluid 34 includes the compound of Formula [B] according to the above, and the second fluid 38 includes the compound of Formula [C] according to the above.

In another embodiment, the first fluid 34 includes the prepolymer of Formula [D] according to the above, and the second fluid 38 includes a crosslinking agent (i.e., a chain extending catalyst), such as for example, glycerol.

When the plunger(s) 40 of the multi-barrel syringe 30 is (are) pressed, the pressure forces the first fluid 34 and the second fluid 38 to flow downstream. At or near the tip 42 of the multi-barrel syringe 30, the first fluid 34 and the second fluid 38 are mixed together.

According to an embodiment, the first fluid 34 and the second fluid 38 are mixed together the outside of the multi-barrel syringe 30.

According to an embodiment, the first fluid 34 and the second fluid 38 are mixed together downstream of the first barrel chamber 32 and the second barrel chamber 36 of the multi-barrel syringe 30.

According to an embodiment, the mixing together of the first liquid 34 and the second liquid 38 is not outside of the multi-barrel syringe 30.

According to an embodiment, the tip 42 is a mixing tip 42, which may or may not be a separable component, and the first fluid 34 and the second fluid 38 are mixed together as they flow through the mixing tip 42.

FIG. 12 shows a nonlimiting exemplary embodiment of a method 50 of producing an expandable biocompatible polymer material. In the first step 52, at least one polymer is obtained, wherein the at least one polymer comprises at least one monomer unit or a prepolymer such as for example one or more chosen from: at least one lactide unit, at least one glycolide unit, at least one caprolactone unit, or any combination thereof. In the second step 54, at least one compound comprising at least one isocyanate group is obtained.

According to an embodiment, in the first step 52, the monomer unit is a compound of Formula [B] and in the second step 54, the compound includes that of Formula [C].

According to an embodiment, in the first step 52, the prepolymer is a compound of Formula [D] and in the second step 54, the compound includes a crosslinking agent (i.e., a chain extending catalyst), such as for example, glycerol. In the next step 56, the at least one polymer with the at least one at least one compound comprising the at least one isocyanate group are mixed together such that they react to form a biocompatible polymer material. The biocompatible polymer material can be used to fill at least one portion of a cavity or an empty space, such as for example, a tooth or a tooth canal.

In some embodiments of the methods for making the polymer material, the method does not include using a surfactant. That is, the process of making the polymer material does not require any surfactant. Examples of the surfactants include, but are not necessarily limited to one or more of the following: silane, sodium lauryl sulphate (SLS), cocamidopropyl betaine (tego betain) and sodium methyl cocoyl taurate (adinol). In some embodiments of the methods for making the polymer material, the method does not include using an additive bonding agent. That is, the process of making the polymer material does not require any additive bonding agent. Examples of the bonding agents include, but are not necessarily limited to one or more of an adhesive, an epoxy, a resin, or acetone. In some embodiments of the methods for making the polymer material, the method does not include using both of the surfactant and the bonding agent.

While a number of embodiments of the present disclosure have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

The following aspects are provided as exemplary embodiments of the present disclosure.
Aspect 1. A composition comprising:
   a polyol part comprising:
      a catalyst,
         wherein the catalyst comprises at least one of a base catalyst, a metal catalyst, a quaternary ammonium catalyst, a phosphorus-based catalyst, or any combination thereof,
      a defoamer, and
      modified nanoparticles,
   wherein the composition is an elastomeric polyurethane sealer.
Aspect 2. The composition according to Aspect 1, wherein the polyol part comprises a hydroxyl terminated polyol.
Aspect 3. The composition according to Aspects 1-2, wherein the hydroxyl terminated polyol comprises at least one of a polyether polyol, a polycaprolactone polyol (PCL), a polytetrahydrofuran (PTHF), a polyester polyol, a polycarbonate polyol, or any combination thereof.
Aspect 4. The composition according to Aspects 1-3, wherein the base catalyst comprises at least one of a guanidine-based catalyst, an amidine-based catalyst, or any combination thereof.
Aspect 5. The composition according to Aspects 1-4, wherein the composition comprises 0.3% to 0.7% by weight of the catalyst based on a total composition.
Aspect 6. The composition according to Aspects 1-5, wherein the composition comprises 0.1% to 0.3% by weight of the defoamer based on a total composition.
Aspect 7. The composition according to Aspects 1-6, wherein the defoamer comprises at least a silicone-based defoamer, a non-silicone based defoamer, or any combination thereof.
Aspect 8. The composition according to Aspects 1-7, the silicone-based defoamer comprises Antifoam A concentrate.
Aspect 9. The composition according to Aspects 1-8, the non-silicone based defoamer comprises at least one of a polyether-based defoamer, a fluorosilicone-based defoamer, a mineral oil based defoamer, a fatty acid-based defoamer, an alcohol-based defoamer, an ester-based defoamer, or any combination thereof.
Aspect 10. The composition according to Aspects 1-9, wherein the defoamer is configured to control expansion of the composition.
Aspect 11. The composition according to Aspects 1-10, wherein the modified nanoparticles comprises fatty-acid functionalized particles.
Aspect 12. The composition according to Aspects 1-11, wherein the fatty-acid functionalized particles comprises 5 wt % stearic acid, palmitic acid, and oleic acid.
Aspect 13. The composition according to Aspects 1-12, wherein the modified nanoparticles comprise surface hydroxyl groups configured to react with diisocyanates.
Aspect 14. The composition according to Aspects 1-13, further comprising a diisocyanate part.
Aspect 15. The composition according to Aspects 1-14, wherein the diisocyanate part comprises at least a bio-based diisocyanate, a non-aromatic diisocyanate, an aromatic diisocyanate, or any combination thereof.
Aspect 16. The composition according to Aspects 1-15, wherein the bio-based diisocyanate comprises a L-lysine diisocyanate.
Aspect 17. The composition according to Aspects 1-16, wherein the diisocyanate comprises a moisture scavenger.
Aspect 18. A composition comprising:
   a polyol part comprising:
      a catalyst,
         wherein the catalyst comprises at least one of a base catalyst, a metal catalyst, a quaternary ammonium catalyst, a phosphorus-based catalyst, or any combination thereof,
      defoamer, and
      modified nanoparticles; and
   a diisocyanate part,
      wherein the diisocyanate part and the polyol part are present at a 1:1 molar ratio;
   wherein the composition is an elastomeric polyurethane sealer.
Aspect 19. A composition, comprising:
   a polymer material including a compound of Formula [A]:

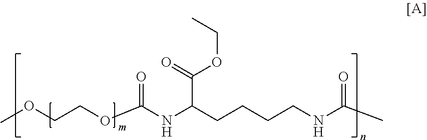

wherein:
   m is 1 to 100 million, and
   n is 2 to 100 million; and
   a radiopaque material,
      wherein the radiopaque material comprises at least one of: gold, platinum, tungsten, platinum-tungsten, palladium, iridium, platinum-iridium, rhodium, tantalum, barium sulfate, bismuth subcarbonate, bismuth oxychloride, bismuth trioxide, a radiopaque metal, a radiopaque alloy, or a radiopaque ceramic, or any combination thereof.
Aspect 20. The composition according to Aspect 19, wherein the composition is a maxillofacial filler.

What is claimed is:

1. A composition comprising:
a polyol part comprising:
a catalyst,
wherein the catalyst comprises at least one of a base catalyst, a metal catalyst, a quaternary ammonium catalyst, a phosphorus-based catalyst, or any combination thereof,
a defoamer, and
modified nanoparticles,
wherein the composition is an elastomeric polyurethane sealer.

2. The composition of claim 1, wherein the polyol part comprises a hydroxyl terminated polyol.

3. The composition of claim 2, wherein the hydroxyl terminated polyol comprises at least one of a polyether polyol, a polycaprolactone polyol (PCL), a polytetrahydrofuran (PTHF), a polyester polyol, a polycarbonate polyol, or any combination thereof.

4. The composition of claim 1, wherein the base catalyst comprises at least one of a guanidine-based catalyst, an amidine-based catalyst, or any combination thereof.

5. The composition of claim 1, wherein the composition comprises 0.3% to 0.7% by weight of the catalyst based on a total composition.

6. The composition of claim 1, wherein the composition comprises 0.1% to 0.3% by weight of the defoamer based on a total composition.

7. The composition of claim 1, wherein the defoamer comprises at least a silicone-based defoamer, a non-silicone based defoamer, or any combination thereof.

8. The composition of claim 7, the silicone-based defoamer comprises Antifoam A concentrate.

9. The composition of claim 7, the non-silicone based defoamer comprises at least one of a polyether-based defoamer, a fluorosilicone-based defoamer, a mineral oil based defoamer, a fatty acid-based defoamer, an alcohol-based defoamer, an ester-based defoamer, or any combination thereof.

10. The composition of claim 1, wherein the defoamer is configured to control expansion of the composition.

11. The composition of claim 1, wherein the modified nanoparticles comprises fatty-acid functionalized particles.

12. The composition of claim 11, wherein the fatty-acid functionalized particles comprises 5 wt % stearic acid, palmitic acid, and oleic acid.

13. The composition of claim 1, wherein the modified nanoparticles comprise surface hydroxyl groups configured to react with diisocyanates.

14. The composition of claim 1, further comprising a diisocyanate part.

15. The composition of claim 14, wherein the diisocyanate part comprises at least a bio-based diisocyanate, a non-aromatic diisocyanate, an aromatic diisocyanate, or any combination thereof.

16. The composition of claim 15, wherein the bio-based diisocyanate comprises a L-lysine diisocyanate.

17. The composition of claim 15, wherein the diisocyanate comprises a moisture scavenger.

18. A composition comprising:
a polyol part comprising:
a catalyst,
wherein the catalyst comprises at least one of a base catalyst, a metal catalyst, a quaternary ammonium catalyst, a phosphorus-based catalyst, or any combination thereof,
defoamer, and
modified nanoparticles; and
a diisocyanate part,
wherein the diisocyanate part and the polyol part are present at a 1:1 molar ratio;
wherein the composition is an elastomeric polyurethane sealer.

19. A composition, comprising:
a polymer material including a compound of Formula [A]: 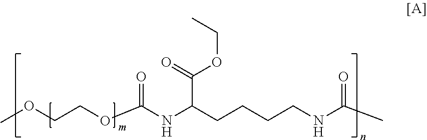
wherein:
m is 1 to 100 million, and
n is 2 to 100 million; and
a radiopaque material,
wherein the radiopaque material comprises at least one of: gold, platinum, tungsten, platinum-tungsten, palladium, iridium, platinum-iridium, rhodium, tantalum, barium sulfate, bismuth subcarbonate, bismuth oxychloride, bismuth trioxide, a radiopaque metal, a radiopaque alloy, or a radiopaque ceramic, or any combination thereof.

20. The composition of claim 19, wherein the composition is a maxillofacial filler.
